(19) United States
(12) Patent Application Publication
(10) Pub. No.: US 2008/0247960 A1
(43) Pub. Date: Oct. 9, 2008

(54) Foaming Tropical Compositions

(75) Inventor: Haoming Yuan, Parsippany, NJ (US)

Correspondence Address: NORRIS, MCLAUGHLIN & MARCUS, 875 THIRD AVE, 18TH FLOOR, NEW YORK, NY, 10022, US

(73) Assignee: RECKITT BENCKISER INC., Parsippany, NJ (US)

(21) Appl. No.: 12/065,333

(22) PCT Filed: Sep. 14, 2006
(86) PCT No.: PCT/GB06/03413

§ 371 Date: Feb. 29, 2008

Related U.S. Application Data
(60) Provisional application No. 60/717,794, filed on Sep. 16, 2005.
(60) Provisional application No. 60/787,865, filed on Mar. 31, 2006.

(51) Int. Cl. A61K 9/12; A61P 31/00

(52) U.S. Cl. 424/43

(57) ABSTRACT

Improved aqueous foaming topical compositions for the application to the human body particularly to the skin which composition provides both a cleaning benefit, and a durable antimicrobial benefit.

The present invention relates to foaming topical compositions particularly adapted for application to human skin. More particularly, the compositions according to the invention are directed to topical compositions which are good foaming and in addition to providing a cleaning benefit, are also intended to provide an antimicrobial benefit.

Topical compositions, per se, are well-known in the cosmetic, dermatological as well as in the pharmaceutical fields. Most topical compositions are intended to provide at least one but generally provide two or more specific benefits after being applied to the human skin. For example, personal care compositions which are primarily intended to be soaps for general cleaning of the human skin such as hand soaps or body wash soaps are well known in the fields of cosmetics and personal care products. While providing a primary cleaning benefit, such personal care compositions frequently also provide ancillary benefits such as moisturizing and nourishing the skin.

The compositions of the present invention are directed to providing at least two primary technical benefits, the first being providing effective cleaning and the second being efficacy in the reduction of undesired microorganisms, particularly in the reduction of both gram positive and/or gram negative microorganisms, especially wherein the second technical benefit is provided over several hours following initial application to a topical surface, e.g., skin, hair, scalp, etc. The present inventive compositions may also provide one or more further ancillary technical benefits, e.g., skin conditioning and/or skin moisturizing. Yet further optional and ancillary benefits may be provided by the presence of one or more for the optional constituents which may be included in formulations or compositions according to the present intervention. These are discussed in more detail hereinafter, particularly with reference to one or more of the examples set forth below.

Accordingly in one aspect of the invention there is provided an improved foaming topical composition for the application to human body particularly to the skin or hair and most preferably to the skin which composition provides both providing effective cleaning and a reduction of undesired microorganisms, particularly in the reduction of both gram positive and/or gram negative microorganisms which may be present on the topical surface of the human body to which the topical compositions have been applied.

According to a second aspect of the invention there is provided a foaming topical composition according to the first aspect of the invention wherein the topical composition provides a reduction of undesired microorganisms at least 30 minutes, preferably at least 45 minutes, more preferably at least 60 minutes after application to the human body.

According to a yet further aspect of the invention is provided a method for the manufacture or production on improved foaming topical composition as set forth herein.

According to a still further aspect of the invention there is provided an improved method for the treatment of the skin as well as other body surface including the hair which method contemplates the application of a cleaning and/or antimicrobially effective amount of the foaming topical composition described herein in order to provide an effective cleaning and/or antimicrobial benefit.

Other features and advantages of the present invention will be apparent from the following detailed description of the invention and from the accompanying claims.

According to one aspect of the present invention there is provided aqueous foaming topical compositions which provides a topical antimicrobial benefit, which compositions comprise:

an anionic surfactant constituent, preferably necessarily comprising one or more anionic sulfate-surfactants;

optionally but preferably a co-surfactant constituent;

a polysaccharide based thickener constituent, preferably a cellulose based thickener constituent, which most preferably a film forming cellulose based thickener constituent;

a cationic Polyquaternium-type polymer;

an antimicrobial constituent which necessarily includes salicylic acid, salicylic acid salt or salicylate in conjunction with at least one further antimicrobially active compound or material which is effective against gram negative and/or gram positive bacteria;

optionally one or more optional constituents which may be used to impart one or more desired esthetic or technical benefits to the topical compositions and which is compatible with the other constituents present in the composition;

wherein the aqueous foaming topical compositions are at an acidic pH, preferably at a pH of about 5 less and further wherein the compositions exhibit a viscosity of at least about least 1000 cps at 25° C. as measured using a Brookfield viscometer, Type 3 spindle, at 6 rpm.

Preferably the aqueous foaming topical compositions of the invention exhibit antimicrobial efficacy both when contacted with a topical surface, e.g., human skin, other body surfaces or other similar substrate, as well as provides a reduction of undesired microorganisms at least 30 minutes, preferably at least 45 minutes, more preferably at least 60 minutes after application to a topical surface, body surface or other similar substrate.

The compositions of the invention necessarily include one or more anionic surfactants and/or salt forms thereof. Examples of anionic surfactants include alcohol sulfates and sulfonates, alcohol phosphates and phosphonates, alkyl ester sulfates, alkyl diphenyl ether sulfonates, alkyl sulfates, alkyl ether sulfates, sulfate esters of an alkylphenoxy polyoxyethylene ethanol, alkyl monoglyceride sulfates, alkyl sulfonates, alkyl ether sulfates, alpha-olefin sulfonates, beta-alkoxy alkane sulfonates, alkyl ether sulfonates, ethoxylated alkyl sulfonates, alkylaryl sulfonates, alkylaryl sulfates, alkyl monoglyceride sulfonates, alkyl carboxylates, alkyl ether carboxylates, alkyl alkoxy carboxylates having 1 to 5 moles of ethylene oxide, alkylpolyglycolethersulfates (containing up to 10 moles of ethylene oxide), sulfosuccinates, octoxynol or nonoxynol phosphates, taurates, fatty taurides, fatty acid amide polyoxyethylene sulfates, acyl glycerol sulfonates, fatty oleyl glycerol sulfates, alkyl phenol ethylene oxide ether sulfates, paraffin sulfonates, alkyl phosphates, isethionates, N-acyl taurates, alkyl succinamates and sulfosuccinates, alkylpolysaccharide sulfates, alkylpolyglucoside sulfates, alkyl polyethoxy carboxylates, and sarcosinates or mixtures thereof.

Further examples of anionic surfactants include water soluble salts or acids of the formula (ROSO₃)ₓM or (RSO₃)ₓM wherein R is preferably a C₆-C₂₄ hydrocarbyl, preferably an alkyl or hydroxyalkyl having a C₁₀-C₂₀ alkyl component, more preferably a C₁₂-C₁₈ alkyl or hydroxyalkyl, and M is H or a mono-, di- or tri-valent cation, e.g., an alkali metal cation (e.g., sodium, potassium, lithium), or ammonium or substituted ammonium (e.g., methyl-, dimethyl-, and trimethyl ammonium cations and quaternary ammonium cations, such as tetramethyl-ammonium and dimethyl piperidinium cations and quaternary ammonium cations derived from alkylamines such as ethylamine, diethylamine, triethylamine, and mixtures thereof, and the like) and x is an integer, preferably 1 to 3, most preferably 1. Materials sold under the Hostapur and Biosoft trademarks are examples of such anionic surfactants.

Still further examples of anionic surfactants which may find use in the invention compositions include alkyl-diphenyl-ethersulphonates and alkyl-carboxylates. Other anionic surfactants can include salts (including, for example, sodium, potassium, ammonium, and substituted ammonium salts such as mono-, di- and triethanolamine salts) of soap, C₆-C₂₀ linear alkylbenzenesulfonates, C₆-C₂₂ primary or secondary alkanesulfonates, C₆-C₂₄ olefinsulfonates, sulfonated polycarboxylic acids prepared by sulfonation of the pyrolyzed product of alkaline earth metal citrates, C₆-C₂₄ alkylpolyglycolethersulfates, alkyl ester sulfates such as C₁₄₋₁₆ methyl ester sulfates; acyl glycerol sulfonates, fatty oleyl glycerol sulfates, alkyl phenol ethylene oxide ether sulfates, paraffin sulfonates, alkyl phosphates, isethionates such as the acyl isethionates, N-acyl taurates, alkyl succinamates and sulfosuccinates, monoesters of sulfosuccinate (especially saturated and unsaturated C₁₂-C₁₈ monoesters) diesters of sulfosuccinate (especially saturated and unsaturated C₆-C₁₄ diesters), acyl sarcosinates, sulfates of alkylpolysaccharides such as the sulfates of alkylpolyglucoside, branched primary alkyl sulfates, alkyl polyethoxy carboxylates such as those of the formula RO(CH₂CH₂O)ₖCH₂COO⁻M⁺ wherein R is a C₈-C₂₂ alkyl, k is an integer from 0 to 10, and M is a soluble salt-forming cation.

Anionic compounds which function both as surfactants and which also act as a hydrotrope may be included as well as either as part of the anionic surfactant constituent or as a co-surfactant as described hereinafter. Exemplary hydtropes include, inter alia, benzene sulfonates, naphthalene sulfonates, C₁-C₁₁ alkyl benzene sulfonates, naphthalene sulfonates, C₅-C₁₁ alkyl sulfonates, C₆-C₁₁ alkyl sulfates, alkyl diphenyloxide disulfonates, and phosphate ester hydrotropes. The hydrotropic compounds of the invention are often provided in a salt form with a suitable counterion, such as one or more alkali, or alkali earth metals, such as sodium or potassium, especially sodium. However, other water soluble cations such as ammonium, mono-, di- and tri-lower alkyl, i.e., C₁₋₄ alkanol ammonium groups can be used in the place of the alkali metal cations. Exemplary alkyl benzene sulfonates include, for example, isopropylbenzene sulfonates, xylene sulfonates, toluene sulfonates, cumene sulfonates, as well as mixtures thereof. Exemplary C₅-C₁₁ alkyl sulfonates include hexyl sulfonates, octyl sulfonates, and hexyl/octyl sulfonates, and mixtures thereof. Particularly useful hydrotrope compounds include benzene sulfonates, o-toluene sulfonates, m-toluene sulfonates, and p-toluene sulfonates; 2,3-xylene sulfonates, 2,4-xylene sulfonates, and 4,6-xylene sulfonates; cumene sulfonates, wherein such exemplary hydrotropes are generally in a salt form thereof, including sodium and potassium salt forms.

Desirably the anionic surfactant constituent of the inventive compositions necessarily comprise one or more anionic sulfate surfactants which provides good foaming when used, and which is resistant to being rinse off from the skin or other topical surface to which it is applied. By way of non-limiting example especially preferred anionic surfactants which provide such functions are alkyl sulfates especially alkyl ether sulfates, particularly C₈-C₁₈ alkyl ether sulfates which have been observed by the present inventor to be more resistant to being rinsed off a topical surface to which they have been applied than many other anionic surfactants which are commonly used in personal cleaning compositions known to the art, including alpha-olefin sulfonates, particularly C₁₂-C₁₆ olefin sulfonates such as sodium lauryl sulfate. Such foaming anionic surfactants, particularly the preferred alkyl ether sulfates exhibit high foaming and good lathering to the inventive compositions. As such it is highly preferred that the anionic surfactant constituent comprise one or more anionic surfactants based on alkyl sulfates, particularly alkyl ether sulfates and especially one or more of those as described in the following Examples. Desirably a major proportion of the anionic surfactant constituent consists of one or more alkyl sulfates especially alkyl ether sulfates, preferably at least 60% wt., more preferably at least 70% wt, and yet more preferably at least 75% wt. of the anionic surfactant constituent comprises alkyl sulfates especially alkyl ether sulfates. In certain particularly preferred embodiments the anionic surfactant constituent comprises at least 90% wt., more preferably the anionic surfactant constituent consists essentially of one or more alkyl sulfates especially one or more alkyl ether sulfates

The anionic surfactant constituent may be present in any effective amount, and is preferably present in amounts of from 1% wt. to 30% wt. based on the total weight of the composition of which they form a part. Preferably however the anionic surfactant constituent comprises 4-20% wt., preferably from 5-15% wt. based on the total weight of the foaming topical composition of which they form a part.

The compositions of the invention may include, and preferably do include one or more further surfactants selected from nonionic, amphotheric and zwitterionic surfactants as co-surfactants to the anionic surfactant constituent. In certain preferred embodiments a co-surfactant constituent is necessarily present.

Exemplary useful nonionic surfactants are those which include a hydrophobicic base portion, such as a long chain alkyl group or an alkylated aryl group, and a hydrophilic chain portion comprising a sufficient number of ethoxy and/or propoxy moieties to render the nonionic surfactant at least partially soluble or dispersible in water. By way of non-limiting example, such nonionic surfactants include ethoxylated alkylphenols, ethoxylated and propoxylated fatty alcohols, polyethylene glycol ethers of methyl glucose, polyethylene glycol ethers of sorbitol, ethylene oxidepropylene oxide block copolymers, ethoxylated esters of fatty (C₆-C₂₄) acids, condensation products of ethylene oxide with long chain amines or amides, and mixtures thereof. Further exemplary nonionic surfactants include, but are not limited to: methyl gluceth-10, PEG-20 methyl glucose distearate, PEG-20 methyl glucose sesquistearate, C₁₁-C₁₅ pareth-20, ceteth-8, ceteth-12, dodoxynol-12, laureth-15, PEG-20 castor oil, polysorbate 20, steareth-20, polyoxyethylene-10 cetyl ether, polyoxyethylene-10 stearyl ether, polyoxyethylene-20 cetyl ether, polyoxyethylene-10 oleyl ether, polyoxyethylene-20 oleyl ether, an ethoxylated nonylphenol, ethoxylated octylphenol, ethoxylated dodecylphenol, or ethoxylated fatty (C₆-C₂₂) alcohol, including 3 to 20 ethylene oxide moieties, polyoxyethylene-20 isohexadecyl ether, polyoxyethylene-23 glycerol laurate, polyoxyethylene-20 glyceryl stearate, PPG-10 methyl glucose ether, PPG-20 methyl glucose ether, polyoxyethylene-20 sorbitan monoesters, polyoxyethylene 80 castor oil, polyoxyethylene-15 tridecyl ether, polyoxyethylene-6 tridecyl ether, laureth-2, laureth-3, laureth-4, PEG-3 castor oil, PEG 600 dioleate, PEG 400 dioleate, and mixtures thereof. Other nonionic surfactants, although not specifically disclosed herein but known to the art may also be used. The nonionic surfactants may be present as single compounds or as mixtures of two or more nonionic surfactant compounds.

Exemplary useful amphoteric surfactants include derivatives of secondary and tertiary amines having aliphatic radicals that are straight chain or branched, and wherein one of the aliphatic substituents contains from about 8 to 18 carbon atoms and at least one of the aliphatic substituents contains an anionic water-solubilizing group, e.g., a carboxy, sulfonate, or a sulfate group. Non-limiting examples of compounds falling within this description include: sodium 3-(dodecylamino)propionate, sodium 3-(dodecylamino)propane-1-sulfonate, sodium 2-(dodecylamino)ethyl sulfate, sodium 2-(dimethylamino)octadecanoate, disodium 3-(N carboxymethyldodecylamino)propane-1-sulfonate, disodium octadecyliminodiacetate, sodium 1-carboxymethyl-2-undecylimidazole, and sodium N,N-bis(2-hydroxyethyl)-2-sulfato-3-dodecoxypropylamine. Further exemplary useful amphoteric surfactants include sarcosinates and taurates, amide sulfosuccinates, and betaines including phosphobetaines. Further amphoteric surfactants, although not specifically elucidated herein but known to the art may also be used.

When a co-surfactant constituent is present, desirably it comprises one or more betaines. Exemplary useful betaine surfactants which may be represented by the general formula:

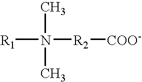

wherein: R₁ is an alkyl group containing from 8 to 18 carbon atoms, or the amido radical which may be represented by the following general formula:

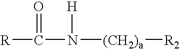

wherein: R is an alkyl group having from 8 to 18 carbon atoms,
a is an integer having a value of from 1 to 4 inclusive, and R₂ is a C₁-C₄ alkylene group.

Examples of preferred betaines are dodecyl dimethyl betaine, cetyl dimethyl betaine, dodecyl amidopropyldimethyl betaine, tetradecyldimethyl betaine, tetradecylanidopropyldimethyl betaine, dodecyldimethylammonium hexanoate and particularly cocoamidopropyl betaine.

As noted previously, in certain preferred embodiments a co-surfactant constituent is necessarily present, and in certain particularly preferred embodiments a betaine surfactant is necessarily present. When present, the co-surfactant constituent may be present in any effective amount, and are preferably present in amounts of from 0.01% wt. to 10% wt. based on the total weight of the composition of which they form a part. Preferably however the co-surfactant constituent comprises 0.1-8% wt., preferably from 0.5-5% wt. based on the total weight of the topical composition of which they form a part.

An optional but preferred additional constituent which may be present is a foam booster which improves the foaming characteristics of the anionic surfactant(s) present. Preferred foam boosters are based on one or more alkanolamides which provide composition thickening, foam enhancement, and foam stability and in preferred embodiments of the invention are necessarily present. Exemplary alkanolamides which provide such a foam boosting function include but are not limited to: cocamide MEA, cocamide DEA, soyamide DEA, lauramide DEA, oleamide MIPA, stearamide MEA, myristamide MEA, lauramide MEA, capramide DEA, ricinoleamide DEA, myristamide DEA, stearamide DEA, oleylamide DEA, tallowamide DEA, lauramide MIPA, tallowamide MEA, isostearamide DEA, isostearamide MEA, and mixtures thereof. When present, the one or more alkanolamides are present in amounts of up to about 10% wt., but are preferably included in amounts of from about 0.1-10% wt. based on the total weight of the topical composition of which they form a part.

The inventive compositions necessarily also comprise a polysaccharide based thickener constituent, e.g., cellulose, alkyl celluloses, alkoxy celluloses, hydroxy alkyl celluloses, alkyl hydroxy alkyl celluloses, carboxy alkyl celluloses, carboxy alkyl hydroxy alkyl celluloses, naturally occurring polysaccharide polymers such as xanthan gum, guar gum, locust bean gum, tragacanth gum, or derivatives thereof, polycarboxylate polymers, polyacrylamides, clays, and mixtures thereof.

Preferred for use in the inventive compositions are one or more cellulose, alkyl cellulose or cellulose derivatives including methyl cellulose ethyl cellulose, hydroxymethyl cellulose, hydroxy ethyl cellulose, hydroxy propyl cellulose, carboxy methyl cellulose, carboxy methyl hydroxyethyl cellulose, hydroxypropyl cellulose, hydroxy propyl methyl cellulose, ethylhydroxymethyl cellulose and ethyl hydroxy ethyl cellulose.

Preferably the inventive compositions comprise a cellulose based thickener constituent preferably a cellulose based thickener constituent, which most preferably a film forming cellulose based thickener constituent. What is to be understood by film forming in the context of the cellulose based thickener is that when the compositions of the invention are topically applied to the skin, preferably human skin, the compositions form a film layer, preferably a continuous film layer at the locus of the topical application, and, especially preferably that the topically applied composition resists being rinsed off the skin by a stream or spray of water.

Whereas it is clearly contemplated that a variety of cellulose based thickener constituents may be used, preferably film forming cellulose based thickener constituents include those which when dispersed at a concentration of 2% w/w in deionized water exhibit a viscosity of at least about 40,000 cPs, preferably at least about 45,000 cPs, more preferably of at least about 50,000 cPs, yet more preferably of at least about 55,000 cPs and especially preferably of at least about 60,000 cPs when measured at room temperature (20° C.). Such increasing viscosities are often characteristic of increasing molecular weight of the cellulose based thickener constituent.

The polysaccharide based thickener constituent, particularly the cellulose based thickener constituent may be present in any effective amount, and are preferably present in amounts of from 0.01% wt. to 7.5% wt. based on the total weight of the composition of which they form a part. Preferably however the co-surfactant constituent comprises 0.1-5% wt., preferably from 0.25-3.0% wt. based on the total weight of the topical composition of which they form a part.

The compositions of the invention necessarily include a cationic Polyquatermium-type polymer. Such materials, are, per se, well known to the art of topical compositions. Various grades of such cationic polymers may be used, inter alia: Polyquaternium 1; Polyquaternium 2; copolymers of hydroxyethylcellulose and diallyldimethyl ammonium chloride commercially available as Polyquaternium 4; homopolymers of diallyldimethylammonium chloride commercially available as Polyquaternium 5; dimethyldiallylammonium chloride homopolymer commercially available as Polyquaternium 6; copolymers of diallyldimethylammonium chloride with acrylamide commercially available as Polyquaternium 7; the polymeric quaternary ammonium salt of methyl and steardyl dimethylaminoethyl methacrylate quaternized with dimethyl sulfate commercially available as Polyquaternium 8; the polymeric quaternary ammonium salt of polydimethylaminoethyl methacrylate quaternized with methyl bromide commercially available as Polyquaternium 9; a polymeric quaternary ammonium salt formed from the reaction of hydroxyethyl cellulose with a trimethylammonium substituted epoxide commercially available as Polyquaternium 10; a polymeric quaternary ammonium polymer formed by the reaction of vinyl pyrrolidine and dimethyl aminoethylmethacrylate commercially available as Polyquatemium 11; a polymeric quaternary ammonium salt prepared by the reaction of ethyl methacrylate/abietyl methacrylate/diethylaminoethyl methacrylate copolymer with dimethyl sulfate commercially available as Polyquaternium 12; a polymeric ammonium salt prepared by the reaction of ethyl methacrylate/oleyl methacrylate/diethylaminoethyl methacrylate copolymer with dimethyl sulfate commercially available as Polyquaterinum 12; a polymeric quaternary ammonium salt prepared by the reaction of ethyl methacrylate/oleyl methacryalte/diethylaminoethyl methacrylate copolymer with dimethyl sulfate commercially available as Polyquaternium 13; Polyquaternium 14; the copolymer of methacrylamide and betamethacrylyloxyethyl trimethyl ammonium chloride commercially available as Polyquatemium 15; the polymeric quaternary ammonium salt formed from methylvinylimidazolium chloride and vinylpyrrolidone commercially available as Polyquaternium 16; polymeric quaternary salts prepared by the reaction of adipic acid and dimethylaminopropylamine reached with dichloroethyl ether commercially available as Polyquaternium 17; a polymeric quaternary salt prepared by the reaction of azelaic acid and dimethylaminopropylamine reacted with dichloroethyl ether commercially available as Polquaternium 18; a polymeric quaternary ammonium salt prepared by the reaction of polyvinyl alcohol with 2,3-epoxy-propylamine commercially available as Polyquaternium 19; a polymeric quaternary ammonium salt prepared by the reaction of polyvinyl octadecyl ether with 2,3-epoxypropylamine commercially available as Polyquaternium 20; copolymers of acrylic acid and dimethyldiallylammonium chloride commercially available as Polyquatemium 22; polymeric quaternary ammonium salts of hydroxyethyl cellulose reacted with lauryl dimethyl ammonium-substituted epoxide commercially available as Polyquaternium 24; a block copolymer formed by the reaction of Polyquaternium 2 and Polyquaternium 17 commercially available as Polyquaternium 27; a polymeric quaternary ammonium salt consisting of vinylpyrrolidone and dimethylaminopropyl methacrylamide monomers commercially available as Polyquatemium 28; chitosans reacted with propylene oxide and quaternized with epichlorohydrin commercially available as Polyquatemium 29; Polyquatemium 30; a polymeric quaternary ammonium salt prepared by the reaction of DMAPA acrylates/acrylic acid/acrylonitrogens copolymer with diethyl sulfate commercially available as Polyquatemium 31; Polyquaternium 32; Polyquatemium 33; Polyquaternium 34; Polyquaternium 35; Polyquaternium 36; Polyquatemium 37; polymeric quaternary ammonium salts of the terpolymer of acrylic acid/diallyldimethylammonium chloride/acrylamide commercially available as Polyquaternium 39; Polyquaternium 42; a copolymer of acrylamide, acrylamidopropyltrimonium chloride, 2-amidopropylacrylamide sulfonate and DMAPA polymers commercially available as Polyquatemium 43; a polymeric quaternary ammonium salt consisting of vinylpyrrolidone and quaternized imidazoline monomers commercially available as Polyquatemium 44; Polyquaternium 45; a polymeric quaternary ammonium salt prepared by the reaction of vinylcaprolactam and vinylpyrrolidone with methylvinylimidazolium commercially available as Polyquaternium 46; a polymer quaternary ammonium chloride formed by the polymerization of acrylic acid with methacrylamidopropyl trimethylammonium chloride and methylacrylate commercially available as Polyquaternium 47; a copolymer of methacryloyl ethyl betaine, 2-hydroxyethyl methacrylate and metacyloyl ethyl trimethyl ammonium chloride commercially available as Polyquaternium 48; a copolymer of methacryloyl ethyl betaine, PEG-9 methacrylate and methacryloyl ethyl trimethyl ammonium chloride commercially available as Polyquaternium 49; Polyquaternium 50; Polyquatemium 51; Polyquaternium 52; a copolymer of acrylic acid, acrylamide and methacrylamidopropyltrimonium chloride commercially available as Polyquaternium 53; a polymeric quaternary ammonium salt prepared by the reaction of aspartic acid and C6-C18 alkylamine with dimethylaminopropylamine and sodium chloroacetate commercially available as Polyquaternium 54; a polymeric quaternary ammonium chloride formed by the reaction of vinylpyrrolidone, dimethylaminopropyl methacrylamide and methacryloylaminopropyl lauryldimonium chloride commercially available as Polyquaternium 55; and a polymeric quaternary ammonium salt consisting of isophorone diisocyanate, butylene glycol and dihydroxyethyldimonium methosulfate monomers commercially available as Polyquaternium 56. Each of the foregoing are described in the literature, particularly in the International Cosmetic Ingredient Dictionary and Handbook, Volume 2 (9^{th} Edition, 2002), at pages 1311-1319. Other polyquarternium compounds although not specifically elucidated here may also be utilized in the present inventive compositions. Particularly preferred is Polyquaternium 7.

The one or more cationic Polyquaternium-type polymers may be present in amounts of from about from 0.001-2.5% wt., preferably in amounts from 0.01-2% wt., but are most desirably present in reduced weight percentages from about 0.05-1% wt. based on the total weight of the inventive composition of which they form a part.

The inventive compositions also necessarily include an antimicrobial constituent which necessarily includes salicylic acid or salt form thereof, in conjunction with at least one further antimicrobially active compound or material which is effective against gram negative and/or gram positive bacteria, and which is compatible with the other constituents present in the composition.

The salicylic acid or salt form thereof is provided to the compositions of the invention in an antimicrobially effective amount. It is also contemplated that in addition to or in place of the salicylic acid which is most preferred, salicylates (including octyl, amyl, phenyl, benzyl, menthyl, glyceryl, and dipropyleneglycol esters thereof) may also be included in the inventive compositions and are believed to be effective against gram positive and/or gram negative microorganisms as well.

The inventor has observed that while other organic acids selected from citric acid, lactic acid, and glycolic acid might also provide an antimicrobial benefit when topically applied, such however provide only an immediate benefit and are not particularly suited to providing a more durable antimicrobial benefit as they are more water soluble than salicylic acid and thus are more easily washed off or rinsed off when topically applied than salicylic acid or salicylates.

The salicylic acid, salicylic acid salt and/or salicylate are present in amounts of from about from 0.001-3% wt., preferably in amounts from 0.01-2% wt., but are most desirably present in reduced weight percentages from about 0.05-0.5% wt. based on the total weight of the inventive composition of which they form a part.

In addition to the salicylic acid or salt form thereof, the inventive compositions necessarily include one further antimicrobially active compound or material which is effective against gram negative and/or gram positive bacteria, and which is compatible with the other constituents present in the composition. Exemplary useful compounds and materials which may be used as the further antimicrobially active compound or material include one or more of one or more antimicrobial agents including: pyrithiones (especially zinc pyrithione which is also known as ZPT), dimethyldimethylol hydantoin (Glydant®), methylchloroisothiazolinone/methylisothiazolinone (Kathon CG®, sodium sulfite, sodium bisulfite, imidazolidinyl urea (Germall 115®), diazolidinyl urea (Germaill II®), benzyl alcohol, 2-bromo-2-nitropropane-1,3-diol (Bronopol®), formalin (formaldehyde), iodopropynyl butylcarbamate (Polyphase P100®), chloroacetamide, methanamine, methyldibromonitrile glutaronitrile (1,2-Dibromo-2,4-dicyanobutane or Tektamer®), glutaraldehyde, 5-bromo-5-nitro-1,3-dioxane (Bronidox®), phenethyl alcohol, o-phenylphenol/sodium o-phenylphenol, sodium hydroxymethylglycinate (Suttocide A®), polymethoxy bicyclic oxazolidine (Nuosept C®), dimethoxane, thimerosal dichlorobenzyl alcohol, captan, chlorphenenesin, dichlorophen, chlorobutanol, glyceryl laurate, halogenated diphenyl ethers like 2,4,4′-trichloro-2′-hydroxy-diphenyl ether (Triclosan® or TCS), 2,2′-dihydroxy-5,5′-dibromo-diphenyl ether, phenolic compounds like phenol, 2-methyl phenol, 3-methyl phenol, 4-methyl phenol, 4-ethyl phenol, 2,4-dimethyl phenol, 2,5-dimethyl phenol, 3,4-dimethyl phenol, 2,6-dimethyl phenol, 4-n-propyl phenol, 4-n-butyl phenol, 4-n-amyl phenol, 4-tert-amyl phenol, 4-n-hexyl phenol, 4-n-heptyl phenol, mono- and poly-alkyl and aromatic halophenols such as p-chlorophenol, methyl p-chlorophenol, ethyl p-chlorophenol, n-propyl p-chlorophenol, n-butyl p-chlorophenol, n-amyl p-chlorophenol, sec-amyl p-chlorophenol, n-hexyl p-chlorophenol, cyclohexyl p-chlorophenol, n-heptyl p-chlorophenol, n-octyl p-chlorophenol, o-chlorophenol, methyl o-chlorophenol, ethyl o-chlorophenol, n-propyl o-chlorophenol, n-butyl o-chlorophenol, n-amyl o-chlorophenol, tert-amyl o-chlorophenol, n-hexyl o-chlorophenol, n-heptyl o-chlorophenol, o-benzyl p-chlorophenol, o-benzyl-m-methyl p-chlorophenol, o-benzyl-m, m-dimethyl p-chlorophenol, o-phenylethyl p-chlorophenol, o-phenylethyl-m-methyl p-chlorophenol, 3-methyl p-chlorophenol, 3,5-, dimethyl p-chlorophenol, 6-ethyl-3-methyl p-chlorophenol, 6-n-propyl-3-methyl p-chlorophenol, 6-iso-propyl-3-methyl p-chlorophenol, 2-ethyl-3,5-dimethyl p-chlorophenol, 6-sec-butyl-3-methyl p-chlorophenol, 2-iso-propyl-3,5-dimethyl p-chlorophenol, 6-diethylmethyl-3-methyl p-chlorophenol, 6-iso-propyl-2-ethyl-3-methyl p-chlorophenol, 2-sec-amyl-3,5-dimethyl p-chlorophenol 2-diethylmethyl-3,5-dimethyl p-chlorophenol, 6-sec-octyl-3-methyl p-chlorophenol, p-chloro-m-cresol, p-bromophenol, methyl p-bromophenol, ethyl p-bromophenol, n-propyl p-bromophenol, n-butyl p-bromophenol, n-amyl p-bromophenol, sec-amyl p-bromophenol, n-hexyl p-bromophenol, cyclohexyl p-bromophenol, o-bromophenol, tert-amyl o-bromophenol, n-hexyl o-bromophenol, n-propyl-m,m-dimethyl o-bromophenol, 2-phenyl phenol, 4-chloro-2-methyl phenol, 4-chloro-3-methyl phenol, 4-chloro-3,5-dimethyl phenol, 2,4-dichloro-3,5-dimethylphenol, 3,4,5,6-terabromo-2-methylphenol, 5-methyl-2-pentylphenol, 4-isopropyl-3-methylphenol, para-chloro-meta-xylenol, dichloro meta xylenol, chlorothymol, 5-chloro-2-hydroxydiphenylmethane, resorcinol and its derivatives including methyl resorcinol, ethyl resorcinol, n-propyl resorcinol, n-butyl resorcinol, n-amyl resorcinol, n-hexyl resorcinol, n-heptyl resorcinol, n-octyl resorcinol, n-nonyl resorcinol, phenyl resorcinol, benzyl resorcinol, phenylethyl resorcinol, phenylpropyl resorcinol, p-chlorobenzyl resorcinol, 5-chloro 2,4-dihydroxydiphenyl methane, 4′-chloro 2,4-dihydroxydiphenyl methane, 5-bromo 2,4-dihydroxydiphenyl methane, and 4′-bromo 2,4-dihydroxydiphenyl methane, bisphenolic compounds like 2,2′-methylene bis(4-chlorophenol), 2,2′-methylene bis(3,4,6-trichlorophenol), 2,2′-methylene bis(4-chloro-6-bromophenol), bis(2-hydroxy-3,5-dichlorophenyl) sulphide, and bis(2-hydroxy-5-chlorobenzyl)sulphide, benzoic esters (parabens) like methylparaben, propylparaben, butylparaben, ethylparaben, isopropylparaben, isobutylparaben, benzylparaben, sodium methylparaben, and sodium propylparaben, halogenated carbanilides (e.g., 3,4,4′-trichlorocarbanilides (Triclocarban® or TCC), 3-trifluoromethyl-4,4′-dichlorocarbanilide, 3,3′,4-trichlorocarbanilide, as well as 2,4-dichloro-3,5-m-xylenol (“DCMX”). The phenol based non-cationic antimicrobials are preferred, of which parachlorometacresol (“PCMC”) and especially parachlorometaxylenol (“PCMX”) is particularly preferred for use in the inventive compositions. Also particularly preferred is chlorohexanol.

The one or more one further antimicrobially active compounds or materials are present in amounts of from about from 0.001-3% wt., preferably in amounts from 0.1-2% wt., but are most desirably present from about 0.1-0.5% wt. based on the total weight of the inventive composition of which they form a part.

The inventors have also found that the superior antimicrobial efficacy is provided when the inventive foaming topical compositions are also maintained in a specific acidic pH range, preferably of about 5 or less, preferably when the pH is maintained in the range of from about 2-5, yet more preferably from about 4 to about 5.

While not wishing to be bound by the following, the inventor has surprisingly found that a durable antimicrobial benefit is provided by the inventive compositions described herein. While not wishing to be bound by the following hypothesis, it is believed that the selection of the salicylic acid, salicyclic acid salt and/or salicylate(s) in conjunction with the one or more further antimicrobially active compounds or materials provide both rapid acting kill of undesired microorganisms when topically applied to the human skin, as well as a more durable antimicrobial benefit. The durability of the antimicrobial benefit is in turn believed to be attributable to the selection of the polysaccharide based thickener constituent, especially the cellulose based thickener which may form a film on the human skin after topically applied, further in conjunction with the Polyquaternium type polymer which may also form a film on the human skin after topically applied. The combination of the polysaccharide based thickener constituent with the Polyquaternium type polymer is believed to provide a film which both protects the skin or other topical surface in the form of a flexible barrier and concurrently the remaining salicylic acid, salicyclic acid salt and/or salicylate(s) in conjunction with the one or more further antimicrobially active compounds or materials which have not been rinsed off and which are retained on the skin or other topical surface continue to provide an antimicrobial benefit for some time after the application of the topical composition by a consumer. It is also hypothesized that possibly, the cationic Polyquaternium type polymer also provides a degree of residual antimicrobial protection as well.

Preferably the topical composition of the present invention provides a reduction of undesired microorganisms at least 30 minutes, preferably at least 45 minutes, more preferably at least 60 minutes after application to the human body. Still more preferably the topical composition of the present invention provides a reduction of undesired microorganisms at least 90 minutes, yet more preferably at least 120 minutes, and especially preferably at least 180 minutes after the application of the topical composition by a consumer. Such efficacy in the reduction of undesired microorganisms may be evaluated per the test protocol described hereinafter.

Such a durable antimicrobial benefit is highly advantageous in controlling bodily odors, as well as providing a general perception of freshness consequent upon the use of the inventive composition.

The foaming topical compositions of the invention may include one or more further optional constituents which may be used to impart one or more desired esthetic or technical benefits to the topical compositions. In certain preferred embodiments of the invention, one or more of the following recited optional constituents may be considered as essential constituents according to a particular preferred embodiment. Such optional constituents include additives and adjuvants which are conventional in the cosmetic, pharmaceutical or dermatological field, such as hydrophilic or lipophilic gelling agents, hydrophilic or lipophilic active agents, humectants, opacifiers, preservatives, antioxidants, solvents especially organic solvents, pH adjusting agents, pH buffers, chealating agents, fragrances, fragrances or other materials which provide an aromatherapy benefit, fillers, preservatives, dyestuffs or colorants, and light stabilizers including UV absorbers. The amounts of these various additives and adjuvants are those conventionally used in the field, and, for example, range from 0.01% to 10% of the total weight of the composition.

One optional constituent which may be included in the inventive compositions is a latex. Such are used to provide opacification of the composition. Such are materials which are typically emulsions, dispersions or suspensions of a water insoluble polymer or copolymer in an carrier. The carrier may be aqueous, an aqueous/organic solvent mixture or organic solvent. The latex may be based on a homopolymer, or on copolymer. It is contemplated that the copolymer comprises two or more different monomers which are joined in either a block or random arrangement of the two or more different monomers.

Exemplary copolymers suitable for the latex emulsion include those formed from styrene, alpha-methylstyrene, divinylbenzene, acrylic acid, methacrylic acid, C₁-C₂₀ esters of acrylic acid or methacrylic acid, acrylamide, methacrylamide, maleic acid, vinyl acetate, crotonic acid, vinyl neodecanoate and butanoic acid. Examples of carboxylate type copolymers are the styrene/alkyl acrylate and partially esterified polyacrylic and polymethacrylic salts and free acid forms. Among the foregoing materials are poly(butyl methacrylate), poly(methyl acrylate), poly(methyl methacrylate), poly(acrylic acid/C₁-C₂₀ alkyl acrylate) and poly(methacrylic acid/C₁-C₂₀ alkyl methacrylate). These copolymers may be prepared by polymerization of the respective monomers by traditional oil-in-water or water-in-oil emulsion polymerization techniques. Alternatively, a pseudo latex may be prepared by esterification of preformed polymer with C₁-C₂₀ alkanol. Average diameters of the dispersed polymer may range from about 0.001 micron to about 120 micron, preferably from about 0.01 micron to about 1 micron, optimally from about 0.1 micron to about 0.5 micron.

Number average molecular weight for polymers according to the present invention may range from about 1,000 to about 1,000,000, preferably from about 2,000 to about 500,000, optimally from about 5,000 to about 20,000.

A variety of techniques well-known in the art can be used to prepare latexes of water-insoluble polymer particles. These include, inter alia, batch, semi-continuous and seeded emulsion polymerization techniques.

Particularly preferred latexes useful in the present invention are latexes presently commercially available under the trademark ACUSOL (ex. Rohm & Haas Inc.). The latexes are characterized by pH of about 2 to about 3, having approximately 40% solids in water, with particle size of about 0.1 to about 0.5 micron. Specific ACUSOL. polymers include ACUSOL OP301 described as being a latex of a styrene/acrylate polymer, ACUSOL OP302 described as being a latex of a styrene/acrylate/divinylbenzene copolymer, ACUSOL OP303 described as being a latex of a styrene/acrylamide copolymer, ACUSOL OP305 described as being a latex of a styrene/PEG-10 maleate/nonoxynol-10 maleate/acrylate copolymer and a styrene/acrylate/PEG-10 dimaleate copolymer. Further preferred latexes useful in the present invention include those styrene/polyvinylpyrrolidone co-polymers and styrene/acrylic emulsions. Such include styrene/polyvinylpyrrolidone co-polymers which can be used include, for example, POLECTRON 430 (ex. ISP Technologies, Inc.), as well as sodium styrene/acrylate/divinyl-benzene co-polymer and ammonium nonoxynol-4 sulfate; sodium stytene/PEG-10 maleate/nonoxynol-10 maleate/acrylates co-polymer and ammonium nonoxynol-4 sulfate; styrene/acrylamide co-polymer and ammonium nonoxynol-4 sulfate; styrene/acrylates co-polymer and sodium lauryl sulfate and octoxynol-9; sodium styrene/acrylates co-polymer and sodium lauryl sulfate and tridecath-7; sodium methacrylate/styrene co-polymer and sodium lauryl sulfate and tridecath-7 and sodium lauryl diphenyloxide-disulfonate; and sodium styrene/acrylates co-polymer (ex CSA, Inc., Greenville, S.C.).

When present in a composition, in accordance with certain of the preferred embodiments, the latex may be present in amounts of up to about 5% wt., preferably are present in amounts of from about 0.001% wt. to about 3% wt., preferably are present in amount from about 0.1% wt. to about 1.2% wt, and most preferably are present in amounts of from about 0.1% wt. to about 1% wt., based on the total weight of the topical composition of which it forms a part. Concurrently the amount of the of the water-insoluble polymer present in the latex may range from about 0.01 to about 90%, preferably from about 0.1 to about 60%, optimally from about 10 to about 50% by weight of the latex.

The topical compositions may include one or more preservatives. Exemplary useful preservatives include compositions which comprise parabens, including methyl parabens and ethyl parabens, glutaraldehyde, formaldehyde, 2-bromo-2-nitropropoane-1,3-diol, 5-chloro-2-methyl-4-isothiazolin-3-one, 2-methyl-4-isothiazoline-3-one, and mixtures thereof. One exemplary composition is a combination 5-chloro-2-methyl-4-isothiazolin-3-one and 2-methyl-4-isothiazolin-3-one where the amount of either component may be present in the mixture anywhere from 0.001 to 99.99 weight percent, based on the total amount of the preservative. For reasons of availability, the most preferred preservative are those commercially available preservative comprising a mixture of 5-chloro-2-methyl-4-isothiazolin-3-one and 2-methyl-4-isothiazolin-3-one marketed under the trademark KATHON CG/ICP as a preservative composition (ex. Rohm and Haas Inc.). Further useful preservative compositions include KATHON CG/ICP II (ex. Rohm and Haas Inc.), PROXEL (ex. Zeneca), SUTTOCIDE A (ex. Sutton Laboratories) as well as TEXTAMER 38AD (ex. Calgon Corp.) When present the preservative is included in any amount found to be effective in retarding or inhibiting the grown of undesired microorganisms in the topical compositions, particularly during storage for several months at room temperature. When present in a composition, in accordance with certain of the preferred embodiments, the preservative composition is advantageously present in amounts of up to about 1.5% wt., preferably are present in amounts of from about 0.00001% wt. to about 0.5% wt., and most preferably is present in an amount of from about 0.0001% wt. to 0.1% wt. based on the total weight of the topical composition of which it forms a part.

The topical compositions may include one or more fillers in the form of powders. By way of non-limiting examples these powders include chalk, talc, kaolin, starch, smectite clays, chemically modified magnesium aluminum silicate, organically modified montmorillonite clay, hydrated aluminum silicate, fumed silica, aluminum starch octenyl succinate and mixtures thereof. When present in a composition, in accordance with certain of the preferred embodiments, the one or more fillers may be present in amounts of up to about 5% wt., preferably are present in amounts of from about 0.001% wt. to about 5% wt. based on the total weight of the topical composition of which it forms a part.

The topical compositions may include a fragrance constituent, which may be based on natural and synthetic fragrances and most commonly are mixtures or blends of a plurality of such fragrances, optionally in conjunction with a carrier such as an organic solvent or a mixture of organic solvents in which the fragrances are dissolved, suspended or dispersed. By way of non-limiting example, natural fragrances include the extracts of blossoms (lily, lavender, rose, jasmine, neroli, ylang-ylang), stems and leaves (geranium, patchouli, petitgrain), fruits (anise, coriander, caraway, juniper), fruit peel (bergamot, lemon, orange), roots (nutmeg, angelica, celery, cardamon, costus, iris, calmus), woods (pinewood, sandalwood, guaiac wood, cedarwood, rosewood), herbs and grasses (tarragon, lemon grass, sage, thyme), needles and branches (spruce, fir, pine, dwarf pine), resins and balsams (galbanum, elemi, benzoin, myrrh, olibanum, opoponax) as well as other further extracts such as eugenol and menthol. Menthol may be advantageously included in that it also provides a cooling sensation when topically applied. Animal raw materials, for example civet and beaver, may also be used. Typical synthetic perfume compounds are products of the ester, ether, aldehyde, ketone, alcohol and hydrocarbon type. Examples of perfume compounds of the ester type are benzyl acetate, phenoxyethyl isobutyrate, p-tert.butyl cyclohexylacetate, linalyl acetate, dimethyl benzyl carbinyl acetate, phenyl ethyl acetate, linalyl benzoate, benzyl formate, ethylmethyl phenyl glycinate, allyl cyclohexyl propionate, styrallyl propionate and benzyl salicylate. Ethers include, for example, benzyl ethyl ether while aldehydes include, for example, the linear alkanals containing 8 to 18 carbon atoms, citral, citronellal, citronellyloxyacetaldehyde, cyclamen aldehyde, hydroxycitronellal, lilial and bourgeonal. Examples of suitable ketones are the ionones, alpha-isomethylionone and methyl cedryl ketone. Suitable alcohols are anethol, citronellol, eugenol, isoeugenol, geraniol, linalool, phenylethyl alcohol and terpineol. The hydrocarbons mainly include the terpenes and balsams. However, it is preferred to use mixtures of different perfume compounds which, together, produce an agreeable fragrance. Other suitable perfume oils are essential oils of relatively low volatility which are mostly used as aroma components. Examples are sage oil, chamomile oil, clove oil, melissa oil, mint oil, cinnamon leaf oil, lime-blossom oil, juniper berry oil, vetiver oil, olibanum oil, galbanum oil, labolanum oil and lavendin oil. A further useful material which finds use in the fragrance constituent is farnesol which is the common chemical name for 3,7,11-trimethyldodeca-2,6,10-trienol, which is commercially available from a number of sources and has found use in cosmetic compositions, primarily as a fragrance constituent. While not wishing to be bound by the following, it is suspected that the inclusion of farnesol may improve the antimicrobial efficacy of the compositions when they are topically applied and used in their normal manner. Such advantageously may reduce the presence of malodor causing microorganisms present and thus improve the perception of body “freshness” when the composition is used.

When present in a composition, in accordance with certain of the preferred embodiments, the fragrance constituent may be present in any effective amount such that it can be discerned by a consumer of the topical composition, however is advantageously present in amounts of up to about 0.5%/wt., preferably are present in amounts of from about 0.00001% wt. to about 0.3% wt., and most preferably is present in an amount of from about 0.0001% wt. to 0.25% wt. based on the total weight of the composition of which it forms a part.

The inventive compositions may include one or more colorants, e.g, dyes or pigments which are known to the art be useful in cosmetic or topical compositions which may be used to impart a desired color or tint to the inventive compositions. Any colorant which is compatible with the other constituents forming the topical compositions may be used and such may be present in any amount effective to achieved the desired visual effect. Exemplary colorants include pigments, inter alia, inorganic red pigments, such as iron oxide, iron hydroxide and iron titanate; inorganic brown pigments, such as gamma.-iron oxide; inorganic yellow pigments, such as iron oxide yellow and loess; inorganic black pigments, such as iron oxide black and carbon black; inorganic violet pigments, such as manganese violet and cobalt violet; inorganic green pigments, such as chromium hydroxide, chromium oxide, cobalt oxide and cobalt titanate; inorganic blue pigments, such as Prussian blue and ultramarine blue; lakes of tar pigments; lakes of natural dyes; and synthetic resin powder complexes of the inorganic pigments as recited above. Advantageously one or more colorants may be added in amounts of about 0.001% wt. to about 0.1% by weight, based on the total weight of the composition of which the colorant(s) forms a part.

The topical compositions may comprise one or more humectants, including polyhydric alcohols including polyalkylene glycols as well as alkylene polyols and their derivatives, inter alia, including propylene glycol, dipropylene glycol, polypropylene glycol, polyethylene glycol and derivatives thereof, sorbitol, hydroxypropyl sorbitol, erythritol, threitol, pentaerythritol, xylitol, glucitol, mannitol, hexylene glycol, butylene glycol (e.g., 1,3-butylene glycol), hexane triol (e.g., 1,2,6-hexanetriol), glycerine, ethoxylated glycerine and propoxylated glycerine. Further useful humectants include sodium 2-pyrrolidone-5-carboxylate, guanidine; glycolic acid and glycolate salts (e.g. ammonium and quaternary alkyl ammonium); lactic acid and lactate salts (e.g. ammonium and quaternary alkyl ammonium); aloe vera in any of its variety of forms (e.g., aloe vera gel); hyaluronic acid and derivatives thereof (e.g., salt derivatives such as sodium hyaluronate); lactamide monoethanolamine; acetamide monoethanolamine; urea; and, panthenol. The humectants may be used singly or two or more humectants may be included in topical compositions of the invention. Of the humectants, aloe vera in one or more of its forms is preferred as being a naturally derived product. When present, in accordance with certain of the preferred embodiments, one or more humectants may be included in effective amounts, advantageously from 0.01-2.5% wt., preferably from 0.01-2% wt. based on the total weight of the composition of which it forms a part.

It is recognized that in addition to the foregoing optional humectants, the Polyquaternium-type polymer may also provide a humectant benefit as well.

The aqueous foaming topical compositions of the invention may include one or more constituents, particularly may include one or more essential oils which are selected to provide a so-called “aromatherapy benefit” to the user. Essential oils are complex mixtures of different organic molecules, such as terpenes, alcohols, esters, aldehydes, ketones and phenols. Such essential oils are frequently extracted from naturally occurring botanical sources such as flowers, stems, leaves, roots and barks of aromatic plants. While essential oils may be used singly, it is also common to utilize blends of essential oils in order to provide a conjunctive aroma benefit, and possibly a therapeutic benefit as well.

A variety of essential oils providing an aromatherapy benefit may be incorporated into the topical compositions of the invention either as a single essential oil or as a mixture of two or more essential oils. It is also to be recognized when used, an essential oil providing an aromatherapy benefit may replace all or part of any further fragrance constituent including the fragrance constituents discussed above as many of the essential oils providing an aromatherapy benefit are pungent and odiferous. Such essential oils providing an aromatherapy benefit may be used singly, as blends or mixtures of essential oils, or in combination with other fragrancing constituents which may be synthetically produced or naturally derived, but need not be derived from or contain essential oils per se. Frequently, due to their potency, essential oils are often supplied dispersed in a liquid carrier such as in one or more organic solvents in which the essential oils are dissolved or dispersed.

By way of non-limiting example, exemplary useful essential oils providing an aromatherapy benefit which may find use in the topical compositions of the invention include: Abies Sibirica oil, Amyris Balsamifera oil, Anise oil, Balm Mint oil, Basil oil, Bay oil, Bee Balm oil, Bergamot oil, Birch oil, Bitter Orange oil, Cabbage Rose oil, Calendula Officinalis oil, California Nutmeg oil, Camellia Sinensis oil, Capsicum Frutescers oleoresin, Caraway Oil, Cardamon Oil, Cedarwood Oil, Chamaecyparis Obtusa Oil, Chamomile Oil, Cinnamon Oil, Citronella Oil, Clary Oil, Clove Oil, Cloveleaf Oil, Coriander Oil, Coriander Seed Oil, Cyperus Esculentus Oil, Cypress Oil, Eucalyptus Citriodora Oil, Eucalyptus Globulus Oil, Fennel Oil, Gardenia Florida Oil, Geranium Maculatum Oil, Ginger Oil, Grapefruit Oil, Hops Oil, Hypericum Perforatum Oil, Hyptis Suaveolens Oil, Indigo Bush Oil, Jasmine Oil, Juniperus Conmunis Oil, Juniperus Virginiana Oil, Labdanum Oil, Laurel Oil, Lavandin Oil, Lavender Oil, Lemon Oil, Lemongrass Oil, Leptospermum Scoparium Oil, Lime Oil, Linden Oil, Litsea Cubeba Oil, Lovage Oil, Mandarin Orange Oil, Massoy Bark Oil, Matricaria Oil, Moroccan Chamomile Oil, Musk Rose Oil, Myrrh Oil, Myrtle Oil, Norway Spruce Oil, Nutmeg Oil, Olax Dissitiflora Oil, Olibanum, Opoponax Oil, Orange Flower Oil, Orange Oil, Palmarosa Oil, Parsley Seed Oil, Passionflower Oil, Patchouli Oil, Pelargonium Graveolens Oil, Peppermint Oil, Pine Oil, Pine Tar Oil, Pine Kernel Oil, Pine Oil, Pine Cone Oil, Rosemary Oil, Rose Oil, Rosewood Oil, Rue Oil, Sage Oil, Sambucus Nigra Oil, Sandalwood Oil, Sandarac Gum, Sassafras Officinalei Oil, Sisymbrium Ino Oil, Spearmint Oil, Sweet Marjoram Oil, Sweet Violet Oil, Tar Oil, Thyme Oil, Vetiveria Zizanoides Oil, Wild Mint Oil, Ximenia Americana Oil, Yarrow Oil, Ylang Yang Oil, or any combinations thereof.

Preferred essential oils providing an aromatherapy benefit for use in the topical compositions of the present invention include one or more selected from chamomile oil, lavendin oil, lavender oil, grapefruit oil, lemon oil, line oil, mandarin orange oil, orange flower oil and orange oil. Chamomile oil may be used to promote both a fresh, clean and attractive scent and possibly provide a stress-relaxing benefit to the user of the topical composition. Lavender oil, and lavendin, may be used to promote both a fresh and attractive scent and possibly also provide a stress-relaxing benefit to the user of the topical composition. One or more of grapefruit oil, lemon oil, line oil, mandarin orange oil, orange flower oil and orange oil provide a clean citrus scent and may possibly impart a perceived therapeutic benefit as well when used.

As used in the present invention, these one or more essential oils providing an aromatherapy benefit are present in an amount about 0.00001 wt. % to about 1 wt. %, based on the total weight of the composition. Preferably, the one or more essential oils providing an aromatherapy benefit are present in an amount about 0.00005 wt. % to about 0.75 wt. %, and more preferably about 0.0001 wt. % to about 0.5 wt. % of the total weight of the composition. It is to be understood that these one or more essential oils providing an aromatherapy benefit may be used with our without the optional fragrancing constituent recited previously and may be used wholly or partially in place of said fragrancing constituent.

The topical compositions may include one or more antioxidant constituents. Examples of antioxidants include but are not limited to, water-soluble antioxidants such as sulfhydryl compounds and their derivatives (e.g., sodium metabisulfite and N-acetyl-cysteine), lipoic acid and dihydrolipoic acid, resveratrol, lactoferrin, glutathione, and ascorbic acid and ascorbic acid derivatives (e.g., ascorbyl palmitate and ascorbyl polypeptide). Oil-soluble antioxidants suitable for use in the compositions of this invention include, but are not limited to, butylated hydroxytoluene, retinoids, tocopherols e.g., tocopherol acetate, tocotrienols, and ubiquinone. Natural extracts containing antioxidants suitable for use in the topical compositions of this invention, include but not limited to, extracts containing flavonoids and isoflavonoids and their derivatives, extracts containing resveratrol and the like. Examples of such natural extracts include grape seed, green tea, pine bark, propolis, and the like. When present the total amount of such antioxidants are usually not in excess of 5% wt, preferably are present in amounts of from 0.0001-4% wt. based on the total weight of the topical composition of which it forms a part. In certain preferred embodiments an one or more antioxidants constituents are necessarily present.

Optionally the topical compositions may include one or more vitamins. Examples of vitamins which can be added include vitamin A, such as vitamin A oil, retinol, retinyl acetate and retinyl palmitate; vitamin B, including vitamin B₂ such as riboflavin, riboflavin butyrate and flavin adenine nucleotide, vitamin B₆ such as pyridoxine hydrochloride, pyridoxine dioctanoate and pyridoxine tripalmitate, vitamin B₁₂ and its derivatives, and vitamin B₁₅ and its derivatives; vitamin C, such as L-ascorbic acid, L-ascorbic acid dipalmitic ester, sodium (L-ascorbic acid)-2-sulfate and dipotassium L-ascorbic acid diphosphate; vitamin D, such as ergocalciferol and cholecarciferol; vitamin E, such as alpha-tocopherol, beta-tocopherol, gamma-tocopherol, dl-alpha-tocopheryl acetate, dl-alpha-tocopheryl nicotinate and dl-alpha-tocopheryl succinate. When present, in accordance with certain of the preferred embodiments, one or more vitamins may be included in effective amounts, advantageously from 0.0001-1% wt., preferably from 0.001-0.75% wt. based on the total weight of the composition of which it forms a part.

The topical compositions may include one or more light stabilizers as well as UV absorbers. Such materials are known to be useful in cosmetic or topical compositions and impart a degree of stability to the compositions which may comprise one or more components which may be deleteriously affected when exposed to certain sources of light, e.g., sunlight, fluorescent light sources. Other such materials are known to stabilize or improve the effect of colorants which may be present in the compositions. Any cosmetically acceptable material or compound which provides protection for one or more of the constituents in the inventive compositions from photolytic degradation or photo-oxidative degradation may be used.

Exemplary light stabilizers as well as UV absorbers include: triazines including s-triazine, triazine derivatives e.g. 2,4,6-trianilino-(p-carbo-2′-ethyl-1′-hexyloxy)-1,3,5-triazine, anisotriazine, ethylhexyltriazone, diethylhexylbutamidotriazone, 2,4,6-tris(diisobutyl 4′-aminobenzalmalonate)-s-triazine and octyltriazone; benzotriazoles and derivatives, e.g. drometrizole trisiloxane, ethylenebis(benzotriazolyl)tetramethylbutylphenol; benzophenone compounds and derivatives e.g., 2-hydroxy-4-methoxy benzophenone, 2-hydroxy-4-methoxy-4′-methyl benzophenone, n-hexyl 2-(4-diethylamino-2-hydroxybenzoyl)benzoate and 2,2′-dihydroxy-4-methoxy benzophenone as well as those materials currently marketed under the UVINUL tradename by BASF; sulphonic acid derivatives of benzophenones, e.g., 2-hydroxy-4-methoxybenzophenon-5-sulphonic acid; esters of benzalmalonic acid, e.g., 4-methoxy benzmalonic acid 2-ethylhexyl esters of benzalmalonic acid; benzoxazole derivatives, e.g., 2,4-bis[5-1(dimethylpropyl)benzoxazol-2-yl(4-phenyl)imino]-6-(2-ethylhexyl)imino-1,3,5-triazine; sulphonic acid derivatives of 3-benzylidencamphen, e.g. 4-(2-oxo-3-bornylidenmethyl)-benzene sulphonic acid and 2-methyl-5-(2-oxo-bornyliden) sulphonic acid; cinnamic acid and cinnamic acid amides, esters of cinnamonic acid, e.g., ethylhexyl methoxycinnamate, isopropyl methoxycinnamate, isoamyl methoxycinnamate, DEA methoxycinnamate, diisopropyl methylcinnamate, glyceryl ethylhexanoate dimethoxycinnamate, 4-methoxy cinnamonic acid 2-ethylhexylester, 4-methoxy cinnamonic acid propylester, 4-methoxy cinnamonic acid isoamylester, 2-cyano-3,3-phenyl cinnamonic acid 2-ethylhexylester (octocrylene); propane-1,3-diones, e.g. 1-(4-tert.-butylphenyl)-3-(4′-methoxy-phenyl)-propane-1,3-dion; phenylbenzimidazoles and sulfonated benzimidazoles, e.g., 2-phenylbenzimidazol-5-sulphonic acid, disodium phenyl dibenzimidazole tetrasulfonate; salicylic acid derivatives including esters of salicylic acid, e.g., ethylhexyl salicylate, dipropylene glycol salicylate, TEA salicylate, salicylic acid 2-ethylhexylester, salicylic acid 4-isopropyl benzylester, salicylic acid homomethylester; compounds or derivatives of compounds based on benzylidenecamphor, e.g., 3-benzyliden camphor, 3-benzylidene norcamphor, 4-methylbenzylidenecamphor, benzylidenecamphorsulfonic acid, camphor benzalkonium methosulfate, terephthalylidenedicamphorsulfonic acid, polyacrylamidomethylbenzylidenecamphor and derivatives thereof; 4-aminobenzoic acid and derivatives e.g., 4-(dimethylamino) benzoic acid 2-ethylhexylester, 4-(dimethylamino) benzoic acid 2-octylester and 4-(dimethylamino) benzoic acid amylester. Any of the foregoing materials provided as acids may used in free acid form or as a salt thereof, e.g., an alkali, alkaline earth, ammonium, alkylammonium, alkanolammonium salt form thereof.

Exemplary and preferred such materials which are presently commercially available include one or more of: CIBAFAST H liquid, described to be sodium benzotriazolyl butylphenol sulfonate with Buteth-3 and tributyl citrate; TINOGARD HS described to be sodium benzotriazolyl butylphenol sulfonates; TINOGARD AS described to be bumetrizole; TINOGARD TL described to be benzotriazolyl dodecyl p-cresol; and TINOGARD Q described to be tris(tetramethylhydroxypiperidinol) citrate, all of which are presently commercially available from Ciba Specialty Chemicals (Muttenz, C H.). Particularly preferred such materials are those sold under the UINUL trademark, (ex. BASF), particularly UVINUL MS 40.

When present, the one or more light stabilizers as well as UV absorbers may be included in any effective amount; advantageously such materials are present in amounts of from 0.0001-1% wt., preferably from 0.001-0.25% wt. based on the total weight of the composition of which it forms a part.

In order to adjust the pH of the inventive compositions, one or more pH adjusting agents as well as one or more pH buffers may be included in the compositions in effective amounts. By way of non-limiting example pH adjusting agents include phosphorus containing compounds, monovalent and polyvalent salts such as of silicates, carbonates, and borates, certain acids and bases, tartrates and certain acetates. Further exemplary pH adjusting agents include mineral acids, basic compositions, and organic acids, which are typically required in only minor amounts. By way of further non-limiting example pH buffering compositions include the alkali metal phosphates, polyphosphates, pyrophosphates, triphosphates, tetraphosphates, silicates, metasilicates, polysilicates, carbonates, hydroxides, and mixtures of the same. Certain salts, such as the alkaline earth phosphates, carbonates, hydroxides, can also function as buffers. It may also be suitable to use as buffers such materials as aluminosilicates (zeolites), borates, aluminates and certain organic materials such as gluconates, succinates, maleates, and their alkali metal salts. When present, the pH adjusting agent, especially the pH buffers are present in an amount effective in order to maintain the pH of the inventive composition within a target pH range. Advantageously they may be included in generally minor amounts such as from 0.001-1.5% wt. but desirably are present in amounts from 0.01-1% wt. Exemplary and preferred pH buffers and pH adjusting agents are described with reference to one or more of the following Examples.

The inventive compositions may include one or more chelating agents. Exemplary useful chelating agents include those known to the art, including by way of non-limiting example; aminopolycarboxylic acids and salts thereof wherein the amino nitrogen has attached thereto two or more substituent groups. Preferred chelating agents include acids and salts, especially the sodium and potassium salts of ethylenediaminetetraacetic acid, diethylenetriaminepentaacetic acid, N-hydroxyethylethylenediaminetriacetic acid, and of which the sodium salts of ethylenediaminetetraacetic acid may be particularly advantageously used. Such chelating agents may be omitted, or they may be included in generally minor amounts such as from 0.001-0.5% wt. based on the weight of the chelating agents and/or salt forms thereof. Desirably, such chelating agents are included in the present inventive composition in amounts from 0.01-0.5%/wt., but are most desirably present in reduced weight percentages from about 0.01-0.2% wt.

As is noted above, the compositions according to the invention are aqueous in nature. Water is added to order to provide to 100% by weight of the compositions of the invention. The water may be tap water, but is preferably distilled and is most preferably deionized water or “soft” water. If the water is tap water, it is preferably substantially free of any undesirable impurities such as organics or inorganics, especially minerals salts which are present in hard water which may thus undesirably interfere with the operation of the constituents present in the topical compositions according to the present invention. Water forms a major proportion of the inventive compositions and is necessarily present in amounts of at least 50% wt., preferably in amounts of at least 60% wt., and more preferably are present in amounts of at least 70% wt. based on the total weight of the topical composition of which it forms a part.

The compositions of the invention are viscous and exhibit a viscosity of at least 1000 cps at 25° C. as measured using a Brookfield viscometer, Type 3 spindle, at 6 rpm. Preferably the compositions exhibit viscosities in the range of at least about 1000 cps as measured under these conditions. Yet more preferably the topical compositions of the invention exhibit a viscosity in the range of about 5000 to about 20,000 cps, yet more preferably from about 6,500 to 17,000 cps, and especially preferably from about 7,500-15,500 cps.

In a further aspect, the present invention also contemplates a method for providing a cleaning and a durable antimicrobial benefit to skin or other topical surface which method contemplates the topical application of an aqueous foaming topical composition as described herein in a cleaning and/or antimicrobially effective amount. Preferably according to the foregoing method, a durable antimicrobial benefit is provided to the skin or other topical surface to which the composition has been applied.

While the topical compositions disclosed herein find a primary use in application to the skin to provide a cleaning and a durable antimicrobial benefit thereto and is contemplated as being provided in a dispenser for use in such a treatment, it is to be understood that this is not to be understood as a limiting definition and that other forms and other uses of the present inventive composition, such as face lotion, milky lotion, cream, face cleansing cream, massage materials, liquid toilet soap, as well as in hair care products such as shampoo, rinse or other hair or scalp treatment are expressly contemplated as being within the scope of the present invention. The topical composition of the invention can be formulated as a lotion, a cream or a gel, which may be transparent, translucent or opaque. The composition can be packaged in a suitable container to suit its viscosity and intended use by the consumer. For example, a lotion or cream can be packaged in a bottle, or can be packaged with a propellant in a propellant-driven aerosol device or a may be packaged in a container fitted with a manually operable pump. The inventive composition can be provided and stored in a non-deformable bottle but more preferably is provided in a squeezable container, such as a tube or deformable bottle which provides for easy dispensing of the composition by the consumer. Thus a further aspect of the invention provides a closed container containing the inventive composition as described herein.

It is to be further expressly understood that topical application of the topical composition disclosed herein may be applied to the skin on any part of the body, including the skin on the face, neck, chest, back, arms, axilla, hands, legs, and scalp.

It is contemplated that in use, the consumer dispenses a quantity of the topical composition described herein and applied it to the skin or any other part of the body which has preferably been wetted with water (e.g, rinsed) prior to application of the topical composition. The topical composition may be rubbed into the applied skin or other part of the body by the consumer to generate a lather or foam, and thereafter it is expected that the treated area is rinsed by the consumer under a stream of running water, e.g, in a shower or by immersion into water, e.g, a bath. Thereafter the skin or other parts of the body of the consumer is permitted to air dry or the use of one or more towels to absorb excess moisture is also contemplated. Thus, a further aspect of the invention is directed to the use of the foaming topical composition as described herein.

The following examples below illustrate exemplary formulations as well as preferred embodiments of the invention. It is to be understood that these examples are provided by way of illustration only and that further useful formulations falling within the scope of the present invention and the claims may be readily produced by one skilled in the art without deviating from the scope and spirit of the invention.

### EXAMPLES

A number of topical compositions produced according to the invention described below were produced in according to the following general protocol:

Into a suitably sized vessel open to the atmosphere provided with a conventional laboratory stirrer, a measured amount of deionized or soft water at 65°-70° C. was provided. Thereafter under stirring was first provided the anionic surfactant(s) and the composition was stirred approximately 20 minutes until uniform. Concurrently in a second suitably sized vessel similarly open to the atmosphere and provided with a stirrer was provided a further aliquot of the deionized water or soft water at 65°-70° C. to which was added, under stirring, the cellulose thickener constituent. This second composition was stirred approximately 15-20 minutes or until the mixture was uniform, thereafter sufficient sodium hydroxide was added to adjust the pH to at least 8.0, and stirring was continued for approximately 15-30 minutes. Next, the contents of the second vessel which had cooled to 50°-55° C. was added to the contents of the first vessel under stirring, and stirring continued for approximately 15-20 minutes or until the mixture was uniform. The contents of the vessel were allowed to cool to 45°-50° C., to which was added any co-surfactant under stirring for approximately 15-20 minutes or until the mixture was uniform. The contents of the vessel were allowed to further cool to 40°-45° C., to which was added under stirring the remaining constituents and stirring was continued for approximately 15-30 minutes or until the composition appeared uniform. Finally, when required, the final pH of the compositions were adjusted by the addition of a further suitable amount of sodium hydroxide to adjust the pH of the compositions to the range of 4.0-5.0.

In the following compositions, the constituents were used “as supplied” from their respective suppliers and may constitute less than 100% wt. “actives”, or may have been supplied as constituting 100% wt. “active” of the named compound, as indicated in the following Tables 1 and 2.

In each of the compositions was included deionized water in “quantum sufficient” (q.s.) in order to provide 100 parts by weight of the specific composition

TABLE 1

| | Ex. 1 | Ex. 2 | Ex. 3 | Ex. 4 | Ex. 5 | Ex. 6 | Ex. 7 | Ex. 8 |
|---|---|---|---|---|---|---|---|---|
| hydroxypropylmethylcellulose | 1.0 | 0.95 | 1.0 | 0.95 | 1.0 | 1.0 | 0.9 | 1.15 |
| sodium laureth ether sulfate | 9.57 | 17.0 | 9.57 | 17.0 | 9.57 | 9.57 | 17.0 | 9.57 |
| sodium cumene sulfonate | 3.0 | — | 5.0 | — | 5.0 | 5.0 | — | 5.0 |
| cocoamidopropyl betaine | 1.91 | 4.0 | 1.91 | 4.0 | 1.91 | 1.91 | 4.0 | 1.91 |
| chlorohexanol | 0.3 | 0.3 | 0.3 | 0.3 | 0.3 | 0.3 | 0.3 | 0.3 |
| salicylic acid | 0.3 | 0.3 | 0.3 | 0.3 | 0.3 | 0.3 | 0.3 | 0.3 |
| propylene glycol | 1.0 | 1.0 | 1.0 | 1.0 | 1.0 | 1.0 | 1.0 | 1.0 |
| Polyquaternium-7 | 4.0 | 3.0 | 4.0 | 3.0 | 4.0 | 4.0 | 3.0 | 4.0 |
| tetrasodium EDTA | — | 0.1 | — | 0.1 | — | — | 0.1 | — |
| citric acid | — | 0.55 | — | 0.55 | — | — | 0.55 | — |
| lactic acid | 0.287 | — | 0.287 | — | 0.287 | 0.287 | — | 0.287 |
| sodium citrate | — | 0.6 | — | 0.6 | — | — | 0.6 | — |
| sodium lactate | 2.871 | 1.0 | 2.87 | 1.0 | 2.87 | 2.87 | 1.0 | 2.87 |
| sodium hydroxide | 0.087 | 0.032 | 0.071 | 0.032 | 0.071 | 0.071 | 0.032 | 0.071 |
| glycerine | 0.96 | 0.96 | 0.96 | 0.96 | 0.96 | 0.96 | 0.96 | 0.96 |
| preservative | 0.02 | 0.02 | 0.02 | 0.02 | 0.02 | 0.02 | 0.02 | 0.02 |
| styrene/acrylates copolymer | 0.4 | — | — | 0.40 | — | — | — | 0.40 |
| pearlescent | — | 1.9 | 1.9 | — | 1.9 | 1.9 | 1.9 | — |
| fragrance | 0.2 | 0.15 | 0.2 | 0.15 | 0.2 | 0.15 | 0.2 | 0.2 |
| colorant | — | 0.0012 | — | — | 0.0012 | 0.00025 | — | — |
| UV absorber | — | — | — | — | — | 0.05 | — | — |
| d.i. water | q.s. | q.s. | q.s. | q.s. | q.s. | q.s. | q.s. | q.s. |

| | Ex. 9 | Ex. 10 | Ex. 11 | Ex. 12 | Ex. 13 | Ex. 14 | Ex. 15 | Ex. 16 |
|---|---|---|---|---|---|---|---|---|
| hydroxypropylmethylcellulose | — | 1.0 | 1.1 | 0.85 | 1.0 | — | — | 0.75 |
| hydroxypropylmethylcellulose - 2 | 1.5 | — | — | — | — | 1.5 | 1.0 | — |
| sodium laureth ether sulfate | 9.57 | 13.0 | 13.0 | 17.0 | 18.0 | 18.0 | 17.0 | 17.0 |
| sodium cumene sulfonate | 5.0 | 3.0 | 3.0 | — | — | — | — | — |
| cocoamidopropyl betaine | 1.91 | 1.91 | 3.0 | 4.0 | 5.0 | 5.0 | 4.0 | 4.0 |
| chlorohexanol | 0.3 | 0.3 | 0.3 | 0.3 | 0.3 | 0.3 | 0.3 | 0.3 |
| salicylic acid | 0.3 | 0.3 | 0.3 | 0.3 | 0.3 | 0.3 | 0.3 | 0.3 |
| propylene glycol | 1.0 | 1.0 | 1.0 | 1.0 | 1.0 | 1.0 | 1.0 | 1.0 |
| Polyquaternium-7 | 4.0 | 4.0 | 4.0 | 3.0 | 4.0 | 4.0 | 3.0 | 3.0 |
| tetrasodium EDTA | — | — | — | 0.1 | — | — | — | — |
| disodium EDTA | — | — | — | — | — | — | — | 0.05 |
| citric acid | — | — | — | 0.555 | — | — | — | 0.55 |
| lactic acid | 0.287 | 0.18 | 0.18 | — | 0.28 | 0.28 | 0.28 | — |
| sodium citrate | — | — | — | 0.6 | — | — | — | 0.6 |
| sodium lactate | 2.87 | 1.80 | 1.80 | 1.0 | 2.8 | 2.8 | 2.8 | 1.0 |
| sodium hydroxide | 0.07 | 0.071 | 0.071 | 0.0321 | 0.00125 | 0.35 | 0.002 | — |
| glycerine | 0.96 | 0.96 | 0.96 | 0.96 | 0.96 | 0.96 | 0.96 | 0.96 |
| preservative | 0.02 | 0.02 | 0.02 | 0.02 | 0.02 | 0.02 | 0.02 | 0.02 |
| styrene/acrylates copolymer | — | — | — | — | — | — | — | — |
| pearlescent | — | — | — | 1.9 | 1.5 | 1.5 | — | 1.5 |
| pearlescent - 2 | — | — | — | — | •— | — | 1.5 | — |
| fragrance | — | — | — | 0.15 | 0.2 | 0.2 | 0.2 | 0.2 |
| colorant | — | — | — | 0.00123 | — | — | — | — |
| UV absorber | — | — | — | — | — | — | — | — |
| d.i. water | q.s. | q.s. | q.s. | q.s. | q.s. | q.s. | q.s. | q.s. |

| | Ex. 17 | Ex. 18 | Ex. 19 | Ex. 20 | Ex. 21 | Ex. 22 | Ex. 23 | Ex. 24 | Ex. 25 |
|---|---|---|---|---|---|---|---|---|---|
| hydroxypropylmethylcellulose | 0.95 | 0.8 | 1.05 | 0.9 | 0.6 | 0.5 | 0.8 | 0.8 | 0.85 |
| sodium laureth ether sulfate | 17.0 | 17.0 | 17.0 | 17.0 | 17.0 | 17.0 | 15.6 | 16.0 | 17.0 |
| sodium cumene sulfonate | — | — | — | — | — | — | — | — | — |
| cocoamidopropyl betaine | 4.0 | 4.0 | 4.0 | 4.0 | 4.0 | 4.0 | 4.0 | 4.0 | 4.0 |
| chlorohexanol | 0.3 | 0.3 | 0.3 | 0.3 | 0.3 | 0.3 | 0.3 | 0.3 | 0.3 |
| salicylic acid | 0.3 | 0.3 | 0.3 | 0.3 | 0.3 | 0.3 | 0.3 | 0.3 | 0.3 |
| propylene glycol | 1.0 | 1.0 | 1.0 | 1.0 | 1.0 | 1.0 | 1.0 | 0.9 | 1.0 |
| Polyquaternium-7 | 3.0 | 3.0 | 3.0 | 0.1 | 0.1 | 0.1 | 0.1 | 0.1 | 0.1 |
| tetrasodium EDTA | — | 0.1 | 0.1 | 0.1 | 0.1 | 0.1 | 0.1 | 0.1 | 0.1 |
| disodium EDTA | 0.05 | — | — | — | — | — | — | — | — |
| citric acid | 0.55 | 0.55 | 1.11 | 0.55 | 0.55 | 0.55 | 0.55 | 0.55 | 0.55 |
| lactic acid | — | — | — | — | — | — | — | — | — |
| sodium citrate | 0.6 | 0.6 | 0.6 | 0.6 | 0.6 | 0.6 | 0.6 | 0.6 | 0.6 |
| sodium lactate | 1.0 | 1.0 | 1.0 | 0.4 | 0.5 | 1.0 | 0.4 | 0.1 | 1.0 |
| sodium hydroxide | 0.00125 | 0.041 | 0.041 | 0.04 | 0.031 | 0.031 | 0.039 | 0.046 | 0.031 |
| sodium chloride | — | — | — | 0.28 | 0.08 | 0.18 | 0.34 | 0.38 | 0.02 |
| glycerine | 0.96 | 0.96 | 0.96 | 0.4 | 0.5 | 0.96 | 0.4 | 0.4 | 0.96 |
| preservative | 0.02 | 0.02 | 0.02 | 0.02 | 0.02 | 0.02 | 0.02 | 0.02 | 0.02 |
| styrene/acrylates copolymer | — | — | — | 0.4 | — | — | — | — | — |
| pearlescent | 1.5 | — | — | — | 1.5 | — | — | — | — |
| pearlescent - 2 | — | — | — | — | — | — | — | — | — |
| menthol | — | — | — | — | — | 0.3 | — | — | — |
| farnesol | — | — | — | — | 0.2 | — | — | — | — |
| fragrance | 0.2 | — | — | 0.15 | 0.2 | 0.48 | 0.15 | 0.35 | 0.15 |
| colorant | — | — | — | — | 0.00088 | 0.00137 | 0.0012 | — | 0.00025 |
| UV absorber | — | — | — | — | — | — | — | — | 0.05 |
| d.i. water | q.s. | q.s. | q.s. | q.s. | q.s. | q.s. | q.s. | q.s. | q.s. |
| pH | | | | | | | | 4.36 | 4.37 |

| | Ex. 26 | Ex. 27 | Ex. 28 | Ex. 29 | Ex. 30 |
|---|---|---|---|---|---|
| hydroxypropylmethylcellulose | 0.5 | 0.85 | 1.15 | 1.15 | 0.6 |
| sodium laureth ether sulfate | 17 | 17 | 9.1 | 9 | 14 |
| sodium cumene sulfonate | — | — | — | — | — |
| cocoamidopropyl betaine | 4 | 4 | 4 | 4 | 4 |
| chlorohexanol | 0.3 | 0.3 | 0.3 | 0.3 | 0.3 |
| salicylic acid | 0.3 | 0.3 | 0.3 | 0.3 | 0.3 |
| propylene glycol | 1 | 1 | 1 | 1 | 1 |
| Polyquaternium-7 | 3 | 3 | 3 | 3 | 3 |
| tetrasodium EDTA | 0.1 | 0.1 | 0.1 | 0.1 | 0.1 |
| disodium EDTA | — | — | — | — | — |
| citric acid | 0.55 | 0.55 | 0.55 | 0.55 | 0.55 |
| lactic acid | — | — | — | — | — |
| sodium citrate | 0.6 | 0.6 | 0.6 | 0.6 | 0.6 |
| sodium lactate | 1 | 1 | .04 | 0.4 | 0.5 |
| sodium hydroxide | 0.0312 | 0.031 | 0.0335 | 0.0316 | 0.0306 |
| sodium chloride | — | — | 0.12 | 0.35 | — |
| glycerine | 0.96 | 0.96 | 0.4 | 0.4 | 0.5 |
| preservative | 0.02 | 0.02 | 0.02 | 0.02 | 0.02 |
| styrene/acrylates copolymer | — | — | — | 0.4 | — |
| pearlescent | — | 1.9 | 1.9 | — | 1.5 |
| pearlescent - 2 | — | — | — | — | — |
| menthol | 0.3 | — | — | — | — |
| farnesol | — | — | — | — | 0.2 |
| fragrance | 0.47 | 0.15 | 0.15 | 0.15 | 0.2 |
| colorant | 0.0013 | 0.002 | 0.0012 | — | — |
| UV absorber | — | 0.05 | — | — | — |
| d.i. water | q.s. | q.s. | q.s. | q.s. | q.s. |
| pH | 4.42 | 4.49 | 4.28 | 4.23 | 4.36 |

The identity of the specific constituents used to produce the foregoing Examples is recited on the following Table 2.

TABLE 2

| | |
|---|---|
| hydroxypropylmethylcellulose | hydroxypropylmethylcellulose (100% wt. actives) provided |
| | as METHOCEL J75MSN (ex. Dow Chem. Co.) |
| hydroxypropylmethylcellulose - 2 | hydroxypropylmethylcellulose (100% wt. actives) provided |
| | as METHOCEL K15M (ex. Dow Chem. Co.) |
| sodium laureth ether sulfate | sodium laureth ether sulfate (70% wt. actives) provided as |
| | TEXAPON N-70NA (ex. Cognis) |
| sodium cumene sulfonate | sodium cumene sulfonate (88% wt. actives) provided as |
| | ELTESOL SC pellets (ex. Huntsman) |
| cocoamidopropyl betaine | cocoamidopropyl betaine (30% wt. actives) provided as |
| | EMPIGEN BS/FA (ex. Huntsman) |
| chlorohexanol | chlorohexanol (100% wt. actives) provided as PCMX (ex. |
| | Thomas Swan plc) |
| salicylic acid | salicylic acid (100% wt. actives) provided as powdered |
| | anhydrous salicylic acid (ex. Mallinckrodt) |
| propylene glycol | propylene glycol (100% wt. actives) (ex. Dow Chem. Co.) |
| Polyquaternium-7 | Polyquaternium-7 (9% wt. actives) provided as |
| | MACKERNIUM 007 (ex. McIntyre Group, Singapore) |
| tetrasodium EDTA | tetrasodium EDTA (100% wt. actives) provided as TRILON B |
| | powder (ex. BASF) |
| disodium EDTA | disodium EDTA (100% wt. actives) provided as EDETA BD |
| | powder (ex. BASF) |
| citric acid | citric acid (98-100% wt. actives) provided as anhydrous citric |
| | acid |
| sodium citrate | sodium citrate (98-100% wt. actives) provided as sodium |
| | citrate dehydrate (ex. Hoffman-LaRoche) |
| sodium lactate | sodium lactate (60% wt. actives) provided as PURASAL |
| | S/HQ 60 (ex. PURAC) |
| sodium hydroxide | sodium hydroxide (98-100% wt. actives) provided as |
| | anhydrous sodium hydroxide |
| sodium chloride | sodium chloride (99-100% wt. actives) provided as pure |
| | vacuum dried NaCl |
| glycerine | glycerine (100% wt. actives) provided as PRICERINE 9091 |
| | (ex. Uniquema) |
| preservative | preservative composition containing |
| | methylchloroisothiazolinone and methylisothiazolinone |
| | (proprietary composition)(ex. Rohm & Haas Co.) |
| styrene/acrylates copolymer | opacifier, described as being a latex of a styrene/acrylate |
| | polymer (proprietary composition) provided as ACUSOL |
| | OP301 (ex. Rohm & Haas Co.) |
| pearlescent | pearlescent constituent, a proprietary composition described |
| | as comprising glycol distearate, cocoamide MEA, sodium |
| | laureth ether sulfate, and laureth-10 provided as |
| | EMPIPEARL XA400/X (ex. Huntsman) |
| pearlescent - 2 | pearlescent constituent, described as comprising water, |
| | glycol distearate, cocoamidopropyl betaine and laureth-4 |
| | provided as TEGOPEARL N300 (ex. Degussa) |
| farnesol | 3,7,11-trimethyldodeca-2,6,10-trienol |
| fragrance | fragrance (proprietary composition of its respective supplier) |
| colorant | colorant |
| UV absorber | UV absorber composition, benzophenone-4 (proprietary |
| | composition) provided as UVINUL MS 40 (ex. BASF) |
| d.i. water | d.i. water |

Each of compositions described on Table 1 was at a pH of 4-5, and exhibited a viscosity of from about 1,000 cPs to about 98,000 cPs at 25° C. as measured using a Brookfield viscometer, Type 3 spindle, at 6 rpm.

In certain of the foregoing formulations, addition of a small amount of sodium chloride was found to be advantageous in providing control over the degree of thickening of the final compositions and hence, of the final viscosity of the compositions.

### Antimicrobial Efficacy (I):

The durable topical antimicrobial efficacy of a composition according to the invention was evaluated in accordance with the following general protocol which utilized a culture of Staphylococcus aureus (ATCC 6538) to demonstrate bactericidal activity on a test dermal surface.

First a culture of S. aureus was prepared and grown overnight in AOAC synthetic broth at 35° C.±2.5° C. Next, several sample epidermal surfaces were prepared by obtaining the skin of freshly slaughtered pigs, shaving and cleaning the epidermis of the skin samples, forming them into 1 inch diameter skin samples and sterilizing them by immersing them in 30% v/v ethanol overnight. Thereafter, three 200 μl samples of the composition according to Ex. 17 (three samples of Ex. 17 were used, the first adjusted to a pH of 4.8, a second replicate adjusted to a pH of 4.54, and a third replicate adjusted to a pH of 4.23, were tested) were each directly applied to different prepared epidermal surfaces and rubbed on the surface for 30 seconds using a sterile needle, and allowed to remain on the surface for a total of 5 minutes. Next, each of the epidermal surfaces was tilted to an angle of 45 degrees, and rinsed using approximately 10 ml of sterile water. Each of the treated epidermal surfaces was then placed into a sterile Petri dish, and partially covered leaving a small crack to allow to for the dermal surface to air dry at ambient room temperature (approx. 20° C.).

After drying for 3.5 hours, each of the treated epidermal surfaces was treated with 100 μl of the prepared S. aureus culture having approx. 10⁴ colony forming units/ml, which was allowed to remain in contact with the epidermal surface for 10 minutes at room temperature with the cover of the Petri dish used to partially cover the base of the Petri dish containing the test substrate. Thereafter, each of the test substrates was aseptically transferred to separate large test tubes containing 10 ml D/E broth and the test tube was then vortexed. Thereafter one serial dilution was made from each test substrate in TSC which dilutions were plated according to the EN 13697:2001 test protocols. The prepared plates were supplied with TSA, and incubated at 35° C.±2.5° C. for at least 2 days, thereafter a plate count was made, the number of survivors were compared to an untreated control epidermal sample which was also processed according to the foregoing protocol.

The results of the tested substrates is reported as follows:

TABLE A1

| | Log₁₀ reduction |
|---|---|
| Ex. 17, pH = 4.8 | 1.05 |
| Ex. 17, pH = 4.54 | >1.35 |
| Ex. 17, pH = 4.23 | >1.35 |

As is evident from the foregoing, subsequent to rinsing and drying of each of the tested epidermal substrates, the compositions according to Ex. 17 of the invention provided a pronounced residual antimicrobial benefit.

### Antimicrobial Efficacy (II):

The durable topical antimicrobial efficacy of a composition according to the invention was evaluated in accordance with the following general protocol which utilized a culture of Staphylococcus aureus (ATCC 6538) to demonstrate bactericidal activity on a test dermal surface.

First a culture of S. aureus was prepared and grown overnight in AOAC synthetic broth at 35° C.±2.5° C. Next, several sample epidermal surfaces were prepared by obtaining the skin of freshly slaughtered pigs, shaving and cleaning the epidermis of the skin samples, forming them into 1 inch diameter skin samples and sterilizing them by immersing them in 30% v/v ethanol overnight. Thereafter, 200 μl samples of the compositions according to Examples 24, 26, 27, 28, 29 and 30 were each directly applied to two different prepared epidermal surfaces and rubbed on each surface for 30 seconds using a sterile needle, and allowed to remain on the surface for a total of 5 minutes. Next, each of the epidermal surfaces was tilted to an angle of 45 degrees, and rinsed using approximately 10 ml of sterile water. Each of the treated epidermal surfaces was then placed into a sterile Petri dish, and partially covered leaving a small crack to allow to for the treated and rinsed dermal surface to air dry at ambient room temperature (approx. 20° C.), after which the cover of the Petri dish was closed when an epidermal surface was visibly dry.

After drying for 8 hours, each of the dried epidermal surfaces was treated with 100 μl of the prepared S. aureus culture having approx. 10⁴ colony forming units/ml, which was allowed to remain in contact with the epidermal surface for 10 minutes at room temperature with the cover of the Petri dish partially open. Thereafter, each of the test substrates was aseptically transferred to separate large test tubes containing 10 ml D/E broth and the test tube was then vortexed. Thereafter one serial dilution was made from each test substrate in TSC which dilutions were plated according to the EN 13697:2001 test protocols. The prepared plates were supplied with TSA, and incubated at 35° C.±2.5° C. for at least 2 days, thereafter a plate count was made, the number of survivors were compared to an untreated control epidermal sample which was also processed according to the foregoing protocol.

The foregoing test was repeated three times for each of the tested example formulations, and the mean average results of the observed antimicrobial efficacy is reported on the following table:

TABLE A2

| | Log₁₀ reduction |
|---|---|
| Ex. 24 | >1.59 |
| Ex. 26 | 1.17 |
| Ex. 27 | >1.40 |
| Ex. 28 | 1.13 |
| Ex. 29 | >1.40 |
| Ex. 30 | 1.27 |

As is evident from the foregoing, subsequent to rinsing and drying for 8 hours of each of the tested epidermal substrates, the compositions according to the invention provided a pronounced residual antimicrobial benefit.

### Antimicrobial Efficacy:

Certain of the compositions indicated above in Table 1 were evaluated for their antimicrobial efficacy against various microorganism selected from: Staphylococcus aureus (ATCC 6538), Escherichia coli (ATCC 10536), and Corynebacterium xerosis (ATCC 373) in accordance with the following general protocol which based in part on the “Standard Test for the Assessment of the Rapid Germicidal Activity for Antibacterial Liquid and Bar Soap Products” published by the Soap and Detergent Association (USA) 1995 draft, an on “prEN12054—Chemical Disinfection and Antiseptics—Products for Hygienic and Surgical Handrub and Handwash, Bactericidal Activity—Test Methods and Requirements (Phase 2, Step 1)”.

The specific protocol of the test, as well as materials utilized are set forth in the following detailed description. While no established criteria is considered to be a standard for a “pass” or “fail” determination, it is considered by the inventors that any tested formulation which fails to provide a log₁₀ reduction of less than 1 is considered to be a “fail” score, while any formulation which provided a log₁₀ reduction of 3 or more is considered to be “superior” performing formulations. Formulations which provide a log₁₀ reduction of at least 1 are considered to be a “pass” score, although are less preferable to formulations providing a log₁₀ reduction of 3 or more.

### Testing and Evaluation Protocol

Preparation of Test Medium:

A tryptic sodium chloride solution was produced by providing 1 g of tryptone, pancreatic digest of casein, and 8.5 g of sodium chloride to 1 liter of deionized water.

Tryptic soy agar dispensed as ˜400 ml aliquots.

Neutralization liquid can be any validated mixture such as D/E Broth.

Hard water for product dilution (300 ppm CaC0₃)

A “Solution A” was prepared by dissolving 19.84 g of anhydrous MgCl₂ and 46.24 g of anhydrous CaCl₂ in purified water, which was then dilute to 1 liter in a volumetric flask.

A “Solution B” was prepared by dissolving 35.02 g of NaHCO₃ in purified water and dilute to 1 liter in a volumetric flask.

To 600 ml of sterile purified water was added 6 ml of solution A, and 8 ml of solution B, which was then diluted to 1 liter with purified water using a volumetric flask. The final solution was sterilized by passing through a membrane filter with an effective pore size of 0.45 μm or 0.2 μm. The pH of this solution was between 6.8 and 7.2 at 25° C. Where necessary, the hard water was adjusted using 1N HCL or 1N NaOH.

The prepared Solutions A and B may be stored at 2° C. to 8° C. for up to one month.

Test Organisms

The test organisms used were from the group which included: Staphylococcus aureus (ATCC 6538), Escherichia coli (ATCC 10536), and Corynebacterium xerosis (ATCC 373).

The working cultures used were either a second or third generation subculture on TSA slopes from frozen beads. Subcultures were prepared on TSA slopes and incubated at 35±12.5° C. for 18-24 hours.

Preparation of Test Cultures

At least 2 loopfuls of the working slant were removed and the cells were suspended in approximately 10 ml of TSC and rotated at a revolution rate of 150 min⁻¹ for at least three minutes.

A portion of the suspended cells were pipetted and added to an appropriate volume of TSC (usually 9 ml or more depending on the amount of samples tested). Adjustments were made in order to provide 1.5-5.0×10⁸ cfu ml⁻¹ for each organism using for example the biolog transmittance kit. A typical absorbance range between 45 and 50 (biolog) was used for each organism.

These were the test cultures adjusted to the correct level for each organism.

### Preparation of Sample

All samples were tested at a respective dilution of 50% v/v of the product sample in hard water. As an example, a final testing solution of 50% v/v was achieved when the 1 ml of test culture is added, i.e. 5 ml sample, 4 ml hard water and 1 ml test culture.

Temperature

Test solutions were maintained at a controlled temperature of 37±1° C. in a shaking waterbath unless otherwise stated. The temperature was monitored using a calibrated electronic thermometer.

Testing Procedure

The following steps were performed at 37° C.±1° C.

1. In a 20 ml McCartney bottle was added 4 ml hard water to 5 ml of sample
2. The sample mixture was allowed to reach thermal equilibrium in a waterbath. Also provided to the waterbath maintained at 37° C.±1° C. was a tube of 8 ml D/E broth and 1 ml sterile deionized water for each sample mixture tested.
3. Thereafter 1 ml of test culture was added to each sample mixture and vortexed for 5 seconds, which were thereafter left for 1 minute±10 second contact time.
4. At the conclusion of the contact time the 1 ml of the test mixture was removed to to 9 ml of neutralizer, the resultant inactivated mixture formed a 10⁻¹ dilution of the test reaction mixture (1 ml in 9 ml is the standard neutralization dilution. Where this is not applicable due to insufficient neutralization a 0.1 ml in 9.9 ml neutralization may be used).
5. A 5 minute neutralization period was allowed, and thereafter serial dilutions of 1 in 9 ml TSC were performed in order to enumerate the surviving organisms. Plates were poured with molten TSA.
6. Steps 1 to 5 were repeated using a maximum of 3 samples per 1 minute contact time run and thus a 20 second interval between each.
7. The plated were incubate at 35±2.5° C. for 2-3 days and thereafter the colonies were counted.
8. Optionally the test was repeated on at least two occasions, preferably three occasions using a different inoculum for each.

Counting of Test Cultures

The test cultures were counted as follows.

1. Each organism was diluted to 10⁻⁷ in TSC using appropriate dilutions.
2. Thereafter the 10⁻⁶ and 10⁻⁷ dilutions were plated in duplicate and TSA was poured on.
3. The samples (replicates) were incubated at 35±2.5° C. for 2-3 days and thereafter the colonies were counted.

Validation of Neutralization

The following was conducted for each organism/test substance combination.

1. Neutralization was validated at least once for each new formulation/organism tested.
2. Prior to the test procedure the test culture was diluted using TSC to give a cell concentration of 10³ cfu/ml by performing five ten-fold dilutions in TSC.
3. The test solutions (5 ml sample, 4 ml hard water) and 8 ml D/E broth tube were allowed to thermally equilibrate in the waterbath for 5 minutes.
4. 1 ml of TSC was added to each sample solution, vortexed and left for 1 minute.
5. 1 ml of the test mixture was removed to 8 ml neutralizer, vortexed and left for 5 minutes.
6. 1 ml of the test culture (as per step 2) was added to the neutralized mixture and leave for 5 minutes.
7. The neutralized mixture was diluted using serial dilutions in TSC and plates poured in TSA.
8. Steps 3 to 7 were repeated but, replacing product sample with hard water as a control.
9. The samples were incubated at ±2.5° C. for 2-3 days and thereafter colonies counted.

Neutralization was considered to be effective if the number of organisms in the inactivated product mixture was at least ±0.5 log of that recovered from the control.

### Calculation and Reporting of Test Results

Where possible plates containing between 15 and 300 cfa ml⁻¹ were used to determine colony counts.

The weighted mean is the average of choice, and is used when two dilutions have colonies in the range 15-300 cfa ml⁻¹.
Calculation of Microbiocidal Effect (log₁₀ reduction)

The microbiocidal effect (ME) due to the action of the product over the test contact time at the temperature at which the test was performed is expressed by the formula:

ME=Log₁₀Nc−Log₁₀Nd

where:

Nc=Number of cfu/ml of the test culture count.

Nd=Number of cfu/ml of the sample count.

The ME was calculated to 2 decimal places; the mean-average of the ME values for each of the replicates tested for each specific formulation are reported as the Mean Log₁₀ reduction and reported on the following table. The % reduction was also reported on the following table.

Certain of the compositions disclosed above in Table 1 were evaluated for their antimicrobial efficacy against the foregoing identified challenge organisms in accordance with the foregoing testing protocol and their efficacy was determined, and are reported on the following table.

Antimicrobial Efficacy (1 minute contact time)

| | Challenge microorganism: | | Challenge microorganism: | | Challenge microorganism: | |
|---|---|---|---|---|---|---|
| | Staphylococcus aureus | | Escherichia coli | | Corynebacterium xerosis | |
| Example | Mean Log₁₀ | | Mean Log₁₀ | | Mean Log₁₀ | |
| composition | reduction | % reduction | reduction | % reduction | reduction | % reduction |
| Ex. 20 | >4.37 | >99.99% | 4.27 | >99.99% | >3.91 | >99.9% |
| Ex. 21 | >4.37 | >99.99% | 4.41 | >99.99% | >3.91 | >99.9% |
| Ex. 22 | >4.37 | >99.99% | >4.52 | >99.99% | >3.91 | >99.9% |
| Ex. 23 | >4.37 | >99.99% | >4.52 | >99.99% | >3.91 | >99.9% |
| Ex. 24 | >4.37 | >99.99% | 4.42 | >99.99% | >3.91 | >99.9% |
| Ex. 25 | >4.37 | >99.99% | >4.52 | >99.99% | >3.91 | >99.9% |

As is evident from the results reported above, the example formulations demonstrative of the invention and which were among the preferred embodiments provided rapid reduction (viz., at a 1 minute contact time) reduction of both gram positive and gram negative microorganisms according to the specified test assessment were rated to be “superior” in performance.

While the invention is susceptible of various modifications and alternative forms, it is to be understood that specific embodiments thereof have been shown by way of example which are not intended to limit the invention to the particular forms disclosed; on the contrary the intention is to cover all modifications, equivalents and alternatives falling within the scope and spirit of the invention as expressed in the appended claims.

1. (canceled)

2. (canceled)

3. (canceled)

4. (canceled)

5. (canceled)

6. (canceled)

7. Aqueous, foaming topical compositions which provide a topical antimicrobial benefit, which compositions comprise:
an anionic surfactant constituent, preferably necessarily comprising one or more anionic sulfate surfactants;
optionally but preferably a co-surfactant constituent;
a thickener constituent selected from methyl cellulose ethyl cellulose, hydroxymethyl cellulose, hydroxy ethyl cellulose, hydroxy propyl cellulose, carboxy methyl cellulose, carboxy methyl hydroxyethyl cellulose, hydroxypropyl cellulose, hydroxy propyl methyl cellulose, ethylhydroxymethyl cellulose and ethyl hydroxy ethyl cellulose or derivatives thereof, and preferably said thickener constituent is a film forming thickener constituent;
a cationic Polyquatemium-type polymer;
an antimicrobial constituent which necessarily includes salicylic acid, salicylic acid salt or salicylate in conjunction with at least one further antimicrobially active compound or material which is effective against gram negative and/or gram positive bacteria;
optionally one or more optional constituents which may be used to impart one or more desired esthetic or technical benefits to the topical compositions and which is compatible with the other constituents present in the composition;
wherein the aqueous foaming topical compositions are at an acidic pH, preferably at a pH of about 5 less and further wherein the compositions exhibit a viscosity of at least about least 1000 cps at 25° C. as measured using a Brookfield viscometer, Type 3 spindle, at 6 rpm,
wherein the compositions provide a reduction of undesired microorganisms at least 30 minutes, preferably at least 45 minutes, more preferably at least 60 minutes after application to a topical surface, body surface or other similar substrate.

8. An aqueous foaming topical composition according to claim 1 which comprises a phenol based non-cationic antimicrobial compound as the at least one further antimicrobially active compound.

9. An aqueous foaming topical composition according to claim 1 which comprises parachlorometacresol or parachlorometaxylenol as the at least one further antimicrobially active compound.

10. An aqueous foaming topical composition according to claim 2 which comprises parachlorometacresol or parachlorometaxylenol as the at least one further antimicrobially active compound.

11. An aqueous foaming topical composition according to claim which comprises a betaine co-surfactant.

12. An aqueous foaming topical composition according to claim 2 which comprises a betaine co-surfactant.

13. An aqueous foaming topical composition according to claim 3 which comprises a betaine co-surfactant.

14. A method for providing a cleaning and a durable antimicrobial benefit to skin or other topical surface which method contemplates the topical application of an aqueous foaming topical composition according to any preceding claim.
